(19) United States
(12) Patent Application Publication
(10) Pub. No.: US 2026/0258357 A1
(43) Pub. Date: Sep. 3, 2026

(54) METHOD FOR THE CRYOPRESERVATION OF T REGULATORY CELLS

(71) Applicant: The Board of Trustees of the Leland Stanford Junior University, Stanford, CA (US)

(72) Inventors: Everett Hurteau Meyer, Belmont, CA (US); Anna Pavlova, Redwood City, CA (US)

(21) Appl. No.: 18/878,467

(22) PCT Filed: Jul. 7, 2023
(86) PCT No.: PCT/US2023/027135

§ 371 Date: Dec. 23, 2024

Related U.S. Application Data
(60) Provisional application No. 63/359,521, filed on Jul. 8, 2022.

(51) Int. Cl. C12N 5/0783; A61K 35/17; C12N 5/00

(52) U.S. Cl. CPC C12N 5/0637; A61K 35/17; C12N 5/526; C12N 2501/2302

(57) ABSTRACT

Compositions and methods are provided that relate to the cryopreservation of human Treg cells. The Treg cells can be provided for therapeutic use, e.g. administration to a patient in need thereof. The cryopreservation methods disclosed herein provide for improved Treg viability, Treg counts, and Treg function. In particular, the methods feature short periods of time in culture before and after freezing in order to minimize in vitro changes in the phenotype and function of the cells.

### CROSS REFERENCE TO OTHER APPLICATIONS

This application claims the benefit of U.S. Provisional Application No. 63/359,521, filed Jul. 8, 2022, the contents of which are hereby incorporated by reference in its entirety.

### BACKGROUND

Regulatory T cells (Tregs) are a specialized subpopulation of T cells that act to suppress immune response, thereby maintaining homeostasis and self-tolerance. It has been shown that Tregs are able to inhibit T cell proliferation and cytokine production and play a critical role in preventing autoimmunity. Different subsets with various functions of Treg cells exist. Tregs can be identified by flow cytometry. The most specific marker for these cells is FoxP3, which is localized intra-cellularly. Dysregulation in Treg cell frequency or functions may lead to the development of autoimmune disease. Therapeutic Treg modulation is being developed to treat certain immune disorders, for example to prevent graft v host disease, or allograft rejection.

Promising results of initial studies using Treg as a clinical intervention have increased interest in this type of the cellular therapy. As such, methods of isolation and expansion of Tregs have been studied and optimized, but therapeutic uses could be significantly expanded if reliably cryopreserved cells were available. However, cryopreservation and expansion in culture can have a detrimental effect on Tregs, for example can decrease Treg viability, cause abnormal cytokine secretion, and compromise expression of surface markers essential for proper Treg function and processing. Therefore, optimal strategies and conditions for Treg cryopreservation in conjunction with cell culture, and processing for clinical application are of great clinical interest.

### SUMMARY

Compositions and methods are provided for the cryopreservation of human Treg cells. The cryopreserved Treg cells can be formulated for therapeutic use, e.g. administration to a patient in need thereof. The cryopreservation methods disclosed herein provide for improved Treg viability, Treg counts, and Treg function. In particular, the methods of the disclosure utilize short periods of time in culture before and after freezing, which minimize in vitro changes in the phenotype and function of the cells. The short-term stimulation of primary Tregs in the methods of the disclosure preserves the primary phenotype of Treg, unlike previous ex vivo culture expansion methodologies.

In some embodiments, the disclosed method comprises the steps of: (a) collecting cells from a human donor, e.g. by apheresis; (b) selecting for Tregs from the collected cell population, e.g. by sorting; (c) activating the Tregs in vitro; and (d) cryopreserving the Tregs. Steps (a)-(d) are performed in a period of time less than about 72 hours, less than about 68 hours, less than about 64 hours, less than about 60 hours, from the completion of apheresis to initiation of cryopreservation (freezing). In some embodiments the disclosed invention comprises a method including post-freezing steps of (e) thawing the cryopreserved Tregs, (f) activating the thawed Tregs in vitro, and (g) administering the activated Tregs to a recipient. These post-freezing steps (e)-(g) are performed in a period of time of less than about 72 hours, less than about 68 hours, less than about 64 hours, less than about 60 hours, from thawing to administering to a recipient.

In some embodiments, the methods of the disclosure provide for improved Treg viability, where the viability of the Treg cell population is greater than about 70%, greater than about 75%, greater than about 80%, greater than about 85% after each of the pre-freezing (a)-(c) and post-freezing (e)-(g) steps.

In some embodiments, the methods of the disclosure provide for improved Treg recovery from cryopreservation, where greater than about 40%, greater than about 45%, greater than about 50%, greater than about 55% or more of the initial Treg cells from step (b) are recovered for administration in step (f) to a patient.

In some embodiments a composition of cryopreserved Treg cells is provided, in an acceptable excipient, prepared according to the methods of steps (a)-(d) of the disclosure. In some embodiments a composition of thawed Treg cells is provided in a pharmaceutically acceptable medium, prepared according to the methods of the disclosure of steps (a)-(g).

Disclosed herein, in some embodiments, is a method of treating a human subject in need thereof, comprising administering to said human subject a population of Treg cells cryopreserved by the methods of the disclosure. Conditions where the cells are used may include, for example and without limitation, graft versus host disease (GVHD), type 1 diabetes, autoimmunity, and transplantation tolerance.

The methods of the disclosure can provide cells for genetic transduction of Tregs in a method that is not dependent on culture expansion, and which preserves the primary phenotype of the Tregs. The timing of patient collection and freezing with thaw and activation before transduction allows for genetic transduction to be done in a scheduled manner, avoiding lengthy, expensive in vitro expansion. In some embodiments the genetic transduction is coupled with lymphodepletion of the intended recipient, to allow for in vivo expansion after administration of the cells.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures.

FIG. 1. Treg manufacturing.

FIG. 2. Viability (%) of Treg products after thawing.

FIG. 3. Effect of human serum addition on Treg viability.

FIG. 4. Treg counts over manufacturing process: major cell losses were observed over the media exchange steps (arrow marked spin).

FIG. 5A-5C. Flow Cytometry Research panel results. Panel A. Cells profile on different manufacturing steps (dark blue—final product) Panel B. samples color-code and Panel C. geometric MFI heat map.

FIG. 6. Suppression assay results showed increasing proliferation of responder cells in conditions with higher responders to Treg ratios.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication date, which may need to be independently confirmed.

The present disclosure provides a method of cryopreserving a population of cells comprising a high percentage of Tregs with high viability and high function after thawing. The cell populations are useful for therapeutic purposes by administration to an individual in need thereof. Cryopreserved populations of Tregs can contribute to positive clinical outcomes by, for example, reducing or preventing GVHD in a transplant recipient, improving immune reconstitution in a transplant recipient, etc.

Regulatory T cells (“Tregs”) are a specialized subpopulation of T cells that can suppress activation of the immune system and thereby maintain immune tolerance. There are various types of Tregs, for example, TCRαβ+CD4+ regulatory T cells, which include natural regulatory T cells (nTregs) and induced regulatory T cells (iTregs). nTregs are T cells produced in the thymus and delivered to the periphery as a long-lived lineage of self-antigen-specific lymphocytes. iTregs are recruited from circulating lymphocytes and acquire regulatory properties under particular conditions of stimulation in the periphery. nTregs and iTregs are CD4+CD25+; both can inhibit proliferation of CD4+CD25− T cells in a dose-dependent manner, and both are anergic and do not proliferate upon TCR stimulation. In addition to being positive for CD4 and CD25, Tregs are positive for the transcription factor FOXP3, an intracellular marker. Tregs can be identified or selected based on various marker expression profiles. Non-limiting examples of marker expression profiles that can be used to select Tregs include (1) CD4+CD25+CD127dim, (2) CD4+FOXP3+, (3) CD3+CD4+CD25+, (5) CD3+CD4+CD25+CD127dim, (6) CD3+CD4+CD25+CD127dim FOXP3+, (7) CD3+FOXP3+, (8) CD3+CD4+FOXP3+, (9) CD3+CD4+CD25+FOXP3+, (10) CD3+CD25+FOXP3+, (11) CD3+CD25+CD127dim, (12) CD4+CD25+, (13) CD4+CD25+CD127dimFOXP3+, (14) FOXP3+, CD4+FOXP3+, (15) CD4+CD25+FOXP3+, (16) CD25+FOXP3+, or (17) CD25+CD127dim. In some embodiments, the Treg cells are CD4+CD25+CD127^{lo}FoxP3⁺ Tregs. Selection based on certain expression profiles can be achieved based on extracellular markers and without requiring cell permeabilization, for example, in some embodiments selection is based on selecting for cells having a CD4+CD25+CD127^{lo} phenotype.

A composition for pharmaceutical use can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical composition, i.e. Treg cell population, can be administered for therapeutic purposes. Toxicity and therapeutic efficacy of the cells can be determined according to standard pharmaceutical procedures in cell cultures and/or experimental animals. The data obtained from cell culture and/or animal studies can be used in formulating a range of dosages for humans.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxin, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of cells to administer to a patient to halt or reverse the progression the disease condition as required. Utilizing LD₅₀ animal data, and other information available for the agent, a clinician can determine the maximum safe dose for an individual, depending on the route of administration. Utilizing ordinary skill, the competent clinician will be able to optimize the dosage of a particular therapeutic in the course of routine clinical trials.

The Treg cells can be cultured in vitro under various culture conditions, as disclosed herein. The cell population may be conveniently suspended in an appropriate nutrient medium, such as TexMACS, Iscove's modified DMEM, RPMI-1640, etc., and as disclosed herein are supplemented with human serum. The culture may contain activation agent factors to which the cells are responsive.

### Methods

### Collection

In step (a) of the methods of the disclosure, Treg cells are obtained from a human donor. The cells can obtained from a single donor, or can be from combined donors. A donor and a recipient for the cryopreserved cell population can be allogeneic. A donor and a recipient of the cryopreserved cell population can be HLA matched. A donor and a recipient of the cryopreserved cell population can be HLA mismatched, e.g. mismatched at 1, 2, 3, 4, 5, or 6 of the major HLA alleles. A donor and a recipient of the cryopreserved cell population can be haploidentical.

In some embodiments, the initial cell population is obtained from whole blood, e.g. a peripheral blood product. In some embodiments the initial cell population is obtained from a peripheral blood apheresis product, for example, a mobilized peripheral blood apheresis product. The initial cell population may be obtained from at least one apheresis product, two apheresis products, three apheresis products, four apheresis products, five apheresis products, six apheresis products, or more.

### Selection for Tregs

In step (b) the collected cell population is selected for Tregs. The initial population of cells can be refined by selection for Tregs., for example, peripheral blood or a peripheral blood apheresis product. Selection methods for cell populations can comprise positive or negative selection for the cell population of interest.

Selection methods for cell populations can utilize affinity reagents, including but not limited to an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a fulllength DARPin, a fragment of a DARPin, a full-length fynomer, a fragment of a fynomer, a full-length kunitz domain peptide, a fragment of a kunitz domain peptide, a full-length monobody, a fragment of a monobody, a peptide, or a aptamer. In some embodiments, the affinity reagent is directly conjugated to a detection reagent and/or purification reagent. In some cases, the detection reagent and purification reagent are the same. In other cases, the detection reagent and purification reagent are different. For example, the detection reagent and/or purification reagent can be fluorescent, magnetic, or the like.

Affinity reagents can comprise immunoaffinity reagents, utilizing the binding specificity of antibodies or fragments or derivatives thereof to positively or negatively select for a cell population of interest. Selection methods for cell populations can comprise an affinity agent and a column, such as magnetic activated cell sorting (MACS) with specific antibodies and microbeads, for example using the Miltenyi system (CliniMACs) of columns, antibodies, buffers, preparation materials and reagents. Selection methods for cell populations can comprise fluorescent activated cell sorting (FACS), with cell populations sorted based on staining profiles with one or more fluorescently-conjugated antibodies. Selection methods for cell populations can comprise physical adsorption, for example, physical adsorption of T cells to protein ligands such as lectins.

A population of cells comprising Tregs of the disclosure can be selected based on expression of markers including, without limitation, CD3, CD4, CD25, CD127, and combinations thereof to enrich for Tregs. A population of cells comprising Tregs can be selected using magnetic activated cell sorting (MACS). A population of cells comprising Tregs can be selected using fluorescent activated cell sorting (FACS). A population of cells comprising Tregs can be selected using multiple procedures, for example, multiple MACS selections, multiple FACS selections, or a combination of MACS and FACS selections. For example, a first selection may be performed for expression of CD25, isolating CD25⁺ cells from a hematopoietic cell sample, for example with MACS. A second selection may be performed by contacting the CD25⁺ cells with antibodies specific for CD4 and for CD127, where FACS is used to isolate cells that are CD4⁺CD127^{lo/neg}.

In some embodiments the cells are first enriched by CD25 expression by CliniMACS immunomagnetic selection, followed by flow cytometric sorting for CD4⁺, CD127^{lo/neg} expression. The resulting product is enriched for Tregs defined phenotypically as CD4⁺, CD25⁺, CD127^{lo/neg}, which cells express the transcription activator FoxP3. It is noted that FoxP3 is only detectable intracellularly, requiring fixation and permeabilization of the cells prior to staining and analysis by flow cytometry. Therefore, FoxP3 detection is used only to assess the percentage of Tregs in samples drawn from the final cellular product for infusion.

A population of cells enriched for Tregs can be isolated from whole blood. A population of cells enriched for Tregs can be isolated from a peripheral blood apheresis product. A population of cells enriched for Tregs can be isolated from a population of cells previously enriched and/or depleted for one or more other cell types, e.g., isolated from a population of cells depleted of CD34⁺ cells. In some embodiments, Tregs are isolated from the flow-through fraction of a CD34⁺ MACS selection.

The number of Tregs in an initial or Treg-enriched population of cells can be determined, for example, by flow cytometry, where Tregs can be identified as, for example, CD4⁺CD25⁺CD127^{lo/neg} or CD4⁺FOXP3⁺. Dose calculations can be adjusted based on measures of cell viability measurements, e.g., viability determined via flow cytometry with propidium iodide or 7-AAD, or via trypan blue exclusion.

### Activation

In step (c) the population of cells enriched for Treg cells is activated in vitro for a period of time of at least about 24 hours, and not more than about 48 hours at 37° C. The in vitro activation can be from about 24 hours, from about 26 hours, from about 28 hours, from about 30 hours, from about 32 hours, from about 34 hours from about 36 hours, up to about 48 hours, up to about 46 hours, up to about 44 hours, up to about 42 hours, up to about 40 hours, up to about 38 hours, and may be for about 24, about 30, about 36, about 42, about 48 hours.

For activation the cells are suspended in medium at a concentration of from about 0.9×10⁵, viable cells/ml, from about 1×10⁶, and up to about 2×10⁶; up to about 3×10⁶ and may be at a concentration of from about 1.1×10⁶ to about 2×10⁶ viable cells/ml, e.g. at about 1.1×10⁶; 1.2×10⁶; 1.3×10⁶; 1.4×10⁶; 1.5×10⁶; 1.6×10⁶; 1.7×10⁶; 1.8×10⁶; 1.9×10⁶; 2×10⁶. Media exchange to supplemented culture media can be performed by centrifugation, for example at 300×G for 15 minutes, acceleration 7, deceleration 1.

The medium comprises a suitable cell medium, e.g. TexMACs™, etc., and effective dose of human serum, a T cell activating agent, e.g. TransAct™ (Miltenyi); and IL-2. Each of the reagents is generally GMP grade. Human serum is usually present at a concentration of from about 5% to about 15% vol/vol, e.g. from about 6%, about 7%, about 8% about 9%, about 10% about 11% about 12%, up to about 15%, up to about 14%, up to about 13%, up to about 13%, up to about 12%, and may be present at about 10% concentration. IL-2 is present at a concentration of from about 100 IU/ml up to about 5000 IU/ml, e.g. from about 100 IU, from about 200 IU, from about 300 IU, from about 400 IU, from about 500 IU, up to about 5000 IU, up to about 4000 IU, up to about 3000 IU, up to about 2000 IU, up to about 1000 IU, and may be present at about 500 IU. TransAct™ may be added in accordance with the manufacturers' guideline, e.g. diluted from about 1:20 to about 1:10, and may be 1:17.5, 1:15, 1:12.5, etc.

### Freezing

In step (d) of the method, the activated Treg cells are cryopreserved (freezing and storage). Cryopreservation can comprise addition of a preservative agent (e.g., DMSO) to a suspension of the cells, and gradual cooling of cells in a controlled-rate freezer to prevent osmotic cellular injury during ice crystal formation. Cryopreservation can comprise commercial cryopreservation reagents and materials, for example, Cryobags and CryoStor® CS10. In some embodiments the cells are resuspended in Normosol-R, pH 7.4; with hydroxyethyl starch 30.5%; human serum albumin (HSA) 12%; and DMSO 7.5%.

Cryopreserved cells can be stored for periods of time ranging from hours to years at low temperatures. Cryopreserved cells can be stored at ultralow temperatures, for example, −50° C., −60° C., −70° C., −80° C., −90° C., −100° C., −110° C., −120° C., −130° C., −140° C., −150° C., 160° C., -170° C., −180° C., −190° C., −196° C., or less. Cryopreserved cells can be stored in storage devices comprising liquid nitrogen.

Cells of the disclosure can be cryopreserved for any amount of time. Cells of the disclosure may be cryopreserved for greater than at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or more prior to thawing and administration to a subject.

Cells of the disclosure can be cryopreserved for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or less prior to thawing and administration to a subject.

### Thawing

In step (e) cryopreserved Treg products are thawed for a second round of stimulation prior to release of the Tregs for infusion. Thawing is performed less than about 72 hours prior to infusion, and can be performed in a 37 degree bath, following by dilution with 2× volume of cold normosol +2% HSA. The cells can then be placed in stimulation media and rested without activation overnight.

### Second Activation

In step (f) the sorted Treg cells are cultured in vitro for a period of time of at least about 24 hours, and not more than about 48 hours at 37° C. Post-thaw the cells are placed in a suitable medium comprising human serum and IL-2, in the absence of the T cell activating agent. The medium comprises a suitable cell medium, e.g. TexMACs™, etc., and effective dose of human serum and IL-2. Each of the reagents is generally GMP grade. Human serum may be present at a concentration of from about 5% to about 15% vol/vol, e.g. from about 6%, about 7%, about 8% about 9%, about 10% about 11% about 12%, up to about 15%, up to about 14%, up to about 13%, up to about 13%, up to about 12%, and may be present at about 10% concentration. IL-2 is present at a concentration of from about 100 IU/ml up to about 5000 IU/ml, e.g. from about 100 IU, from about 200 IU, from about 300 IU, from about 400 IU, from about 500 IU, up to about 5000 IU, up to about 4000 IU, up to about 3000 IU, up to about 2000 IU, up to about 1000 IU, and may be present at about 500 IU.

Cells are cultured in this medium for a period of from about 12 to about 24 hours, e.g. from about 12 hours, from about 14 hours, from about 16 hours, from about 18 hours, from about 20 hours, up to about 24 hours, up to about 22 hours, up to about 20 hours, up to about 18 hours, up to about 16 hours.

For activation the cells are suspended in medium at a concentration of from about 0.9×10⁵ viable cells/ml from about 1×10⁶, up to about 2×10⁶; up to about 3×10⁶ and may be at a concentration of from about 1.1×10⁶ to about 2×10⁶ viable cells/ml, e.g. at about 1.1×10⁶; 1.2×10⁶; 1.3×10⁶; 1.4×10⁶; 1.5×10⁶; 1.6×10⁶; 1.7×10⁶; 1.8×10⁶; 1.9×10⁶; 2×10⁶. Media exchange to supplemented culture media can be performed by centrifugation, for example at 300xG for 15 minutes, acceleration 7, deceleration 1.

After the initial culture, a T cell activating agent, e.g. TransAct™ is added to the cells and the cell suspension returned to 37° C., 5% CO₂ for a period of from about 12 to about 24 hours, e.g. from about 12 hours, from about 14 hours, from about 16 hours, from about 18 hours, from about 20 hours, up to about 24 hours, up to about 22 hours, up to about 20 hours, up to about 18 hours, up to about 16 hours.

### Administration

In step (g), after the post-thaw activation period, the cells are separated from the activation medium by centrifugation and resuspended in a suitable infusion medium, including without limitation Normosol-R, pH 7.4 (Hospira, Lake Forest, IL) supplemented with 5% HSA as final preparation for infusion.

A population of cells comprising Tregs administered to a subject can be administered at a dose greater than at least about 1×10⁴, 1×10⁵, 2×10⁵, 3×10⁵, 4×10⁵, 5×10⁵, 6×10⁵, 7×10⁵, 8×10⁵, 9×10⁵, 1×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷, 9.5×10⁷, 1×10⁸, 1×10⁸, 1.5×10⁸, 2×10⁸, 2.5×10⁸, 3×10⁸, 3.5×10⁸, 4×10⁷, 4.5×10⁸, 5×10⁸, 5.5×10⁸, 6×10⁸, 6.5×10⁸, 7×10⁸, 7.5×10⁸, 8×10⁸, 8.5×10⁸, 9×10⁸, 9.5×10⁸, 1×10⁹, or more cells per kg of recipient body weight.

In some embodiments, a population of cells comprising Tregs administered to a subject can be administered at a dose of at most about 1×10⁴, 1×10⁵, 2×10⁵, 3×10⁵, 4×10⁵, 5×10⁵, 6×10⁵, 7×10⁵, 8×10⁵, 9×10⁵, 1×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, 2.5×10, 3×10⁷, 3.5×10⁷, 4×10, 4.5×10⁷, 5×10⁷, 5.5×10⁷, 6×10⁷, 6.5×10⁷, 7×10, 7.5×10⁷, 8×10⁷, 8.5×10⁷, 9×10⁷, 9.5×10⁶, 1×10⁸, 1×10⁸, 1.5×10⁸, 2×10⁸, 2.5×10⁸, 3×10⁸, 3.5×10⁸, 4×10⁷, 4.5×10⁸, 5×10⁸, 5.5×10⁸, 6×10⁸, 6.5×10⁸, 7×10⁸, 7.5×10⁸, 8×10⁸, 8.5×10⁸, 9×10⁸, 9.5×10⁸, 1×10⁹, or less cells per kg of recipient body weight, where Tregs are defined as CD4⁺CD25⁺CD127^{dim}, CD3⁺CD4⁺CD25⁺, CD3⁺CD4⁺CD25 CD127^{dim}, CD3⁺CD4⁺CD25⁺CD127^{dim}FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim}FOXP3⁺, FOXP3⁺, CD4⁺FOXP3⁺, CD4⁺CD25⁺FOXP3⁺, CD25⁺FOXP3⁺, or CD25⁺CD127^{dim}.

A population of cells comprising Tregs of the disclosure can comprise, for example, greater than at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more FOXP3⁺ cells as a percentage of total cells, nucleated cells, or CD45⁺ cells, or CD4⁺CD25⁺CD127^{dim} cells as a percentage of total cells, nucleated cells, or CD45⁺ cells, or as a percentage of total cells, nucleated cells, or CD45⁺ cells.

A population of cells comprising Tregs of the disclosure can have a defined level of contaminating non-Treg cells. For example, greater than at least about 1×10², 2×10², 3×10², 4×10², 5×10², 6×10², 7×10², 8×10², 9×10², 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, or more non-Treg cells per kg of recipient body weight can be present in population of cells comprising Tregs of the disclosure, where non-Treg cells are FOXP3⁻ or CD127^{+/bright}.

In some embodiments, at most about 1×10², 2×10², 3×10², 4×10², 5×10², 6×10², 7×10², 8×10², 9×10², 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, or less non-Treg cells per kg of recipient body weight are present in a population of cells comprising Tregs of the disclosure, where non-Treg cells are FOXP3− or CD127⁺/bright.

A population of cells comprising Tregs of the disclosure can comprise, for example, greater than at least about 0.001%, 0.002%, 0.003%, 0.004%, 0.005%, 0.006%, 0.007%, 0.008% 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, or more non-Treg cells as a percentage of total cells, nucleated cells, or CD45+ cells, where non-Treg cells are FOXP3- or CD127+/bright.

In some embodiments, a population of cells comprising Tregs of the disclosure comprises at most about 0.001%, 0.002%, 0.003%, 0.004%, 0.005%, 0.006%, 0.007%, 0.008% 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, or less non-Treg cells as a percentage of total cells, nucleated cells, or CD45+ cells, where non-Treg cells are FOXP3- or CD127+/bright.

### Conditions for Treatment

Treg cells are useful in a variety of conditions, including, for example, treatment of cancer, in association with hematopoietic stem cell transplantation (HCT), for example to reduce graft v host disease, to reduce allograft rejection, and the like. For example, a population of cells comprising Tregs prepared by the methods of the disclosure can be administered to an HCT recipient subject, and the population of Tregs can serve to reduce or prevent GVHD in the HCT recipient subject.

Graft-versus-host disease (GVHD) is an inflammatory disease that can occur in the allogenic transplant setting. GVHD involves donor cells (graft) attacking recipient cells (host). GVHD can be classified into acute GVHD (aGVHD) and chronic GVHD (cGVHD). aGVHD typically occurs in the first 3 months after transplantation. aGVHD can be life-threatening and can involve, for example, the skin, the intestines, and/or the liver. cGVHD typically occurs after the first 3 months following transplant. cGVHD is a major source of late treatment-related complications, and can be life-threatening. In addition to inflammation, cGVHD can lead to the development of fibrosis, which can result in functional disability.

The early morbidity and mortality associated with acute graft versus host disease (aGVHD) is a major factor limiting the success of HCT, as is the long-term morbidity associated with chronic GVHD (cGVHD). The incidence of aGVHD following allogeneic HCT from an HLA-matched sibling donor (MSD) is 20 to 60%, despite the use of various immunosuppressive agents such as tacrolimus, cyclosporine, methotrexate, mycophenolate, anti-thymocyte globulin and corticosteroids. Approximately one-third of patients who undergo allogeneic HCT using a MSD and a T cell replete graft will develop chronic GVHD.

GVHD severity can be graded, for example, using the Glucksberg grade (I-IV) or the International Bone Marrow Transplant Registry (IBMTR) grading system (A-D). The severity of acute GVHD is determined by an assessment of the degree of involvement of the skin, liver, and gastrointestinal tract. The stages of individual organ involvement are combined with (Glucksberg) or without (IBMTR) the patient's performance status to produce an overall grade, which can have prognostic significance. Grading is important in terms of assessing the response to prophylaxis or treatment, impact upon survival, and association with graft-versus-leukemia effect.

Grade I(A) GVHD is characterized as mild disease, grade II(B) GVHD as moderate, grade III(C) as severe, and grade IV(D) life-threatening. The IBMTR grading system defines the severity of acute GVHD as follows: Grade A: stage 1 skin involvement alone (maculopapular rash over <25 percent of the body) with no liver or gastrointestinal involvement; Grade B: stage 2 skin involvement, stage 1 to 2 gut or liver involvement; Grade C: stage 3 involvement of any organ system (generalized erythroderma; bilirubin 6.1 to 15.0 mg/dL; diarrhea 1500 to 2000 mL/day); Grade D: stage 4 involvement of any organ system (generalized erythroderma with bullous formation; bilirubin >15 mg/dL; diarrhea >2000 mL/day OR pain OR ileus). Patients with moderate to severe GVHD have a significantly higher mortality rate compared with those with milder disease, for example, estimated five year survival for patients with grade III(C) aGVHD is 25%, while for patients with grade IV (D) estimated five year survival is 5%.

Management of GVHD may require immunosuppressive therapy (for example, high dose corticosteroids, prolonged administration of immunosuppressants) or cytotoxic mediations, all of which are associated with toxicity. In many cases, immunosuppressive therapies can fail to effectively treat GVHD, or can result in increased susceptibility to infection, or blunted anti-tumor immunity.

In some embodiments, administration of the cryopreserved Treg cells of the disclosure disclosed herein prevent or reduce GVHD in an HCT recipient subject. For example, the methods disclosed herein can prevent any manifestation of GVHD in a subject receiving HCT.

The methods disclosed herein can prevent, for example, any GVHD of stage 1 or above, any GVHD of stage 2 or above, any GVHD of stage 3 or above, or any GVHD of stage 4 in subjects receiving HCT.

In some embodiments the cryopreserved Tregs are administered to a patient for improving graft tolerance, in combination with an organ graft, e.g. kidney transplantation, heart transplantation, lung transplantation, liver transplantation, and the like. Evidence supports the role of CD4⁺CD25⁺ Foxp3⁺ Tregs in the establishment and maintenance of operational tolerance to allografts. Antigen-specific Tregs are capable of suppressing alloresponses to donor HLA peptides in human kidney transplant recipients. Data from renal liver and lung transplantation in humans showed a high number of circulating and intragraft Tregs in tolerant stable recipients.

### Experimental

### Example 1

### Process Development for Manufacturing Thawed Stimulated

### CD4+CD25+CD127dim/negFoxP3+ Regulatory T-Cells

This disclosure provides for improved post-thaw recovery, viability and efficacy/function of a highly purified CD4⁺CD25⁺CD127^{low}FoxP3⁺ Regulatory T Cells (Treg) product that undergoes cryopreservation and thaw, by establishing a set of uniform steps for the manufacturing.

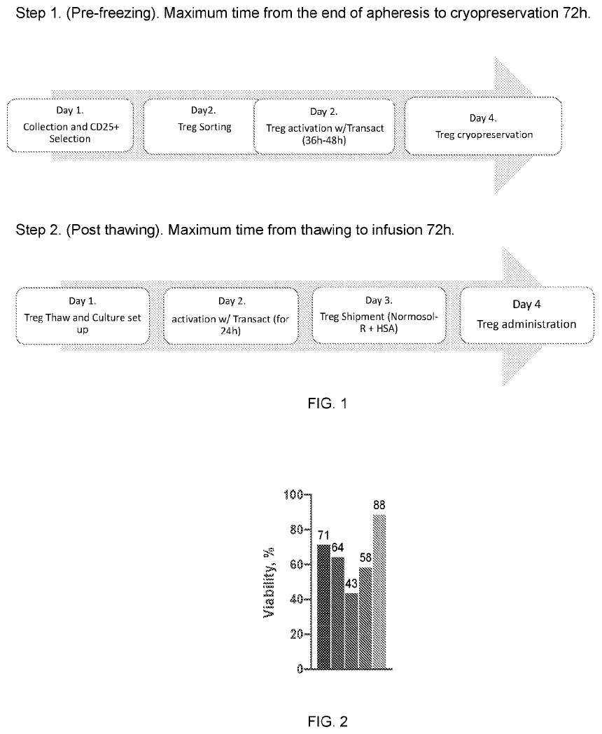

Critical steps for Treg manufacturing have been identified, resulting in the development and testing of a new protocol that resulted in significant improvement in Treg viability (>80% on each step), Treg counts (>50% cells recovered) and function (Flow cytometry assessment: CD4⁺CD62⁺, FoxP3⁺CD25⁺CD127^{low}; suppression assay results). The following protocol includes two steps: pre-freezing Step 1 (maximum length 72 h) and post thawing Step 2 (maximum length 72 h). A schematic overview is presented in FIG. 1. Features of the method include the short overall procedure timeline.

As described in the following sections, the recipient's Tregs are purified using immunomagnetic selection and flow cytometric cell sorting. The cells undergo two rounds of activation by stimulating CD3/CD28 expressing cells. The first round is performed prior to cryopreservation of the Tregs and the second after thawing but prior to distribution of the cells for infusion.

Medium: GMP TexMACS (Miltenyi) (for both pre- and post-freezing culturing) supplemented with GMP OTC Human AB Serum HI GI (Access Biologics), 10% and GMP IL-2 (R&D Systems). Media is exchanged by centrifugation with slow decelleration. Pre-cryopreservation Treg activation uses GMP TransAct (Milyenyi). Post-cryopreservation Treg culturing and activation uses GMP TransAct (Milyenyi).

Pre-freezing. Step 1. Day 1 (step a). Collection and CD25⁺ Selection. Receipt and testing of an apheresis product. Collection and cryopreservation of an aliquot from apheresis product (responder cells for suppression assay.) Labeling with CD25 beads, washing and CD25 selection on CliniMACS. Clinical sorters set up. Overnight Storage of CD25⁺ Enriched fraction at 4 C. Sampling is done for CD25+-Enriched and for QC and research panel.

Step 1. Day 2. (step b) Treg Selection and Cell Culture. Clinical cell sorter preparation. Manual cell viability and count of CD25⁺ Enriched cells. Cell staining and Treg sorting.

Preparation of supplemented culture medium: GMP TexMACS (Miltenyi) medium. GMP OTC Human AB Serum HI GI (Access Biologics) (10% of total volume). (Note: GMP human serum is stored in −80 C. Thaw until no ice is observed, when completely thawed, filter). GMP TransAct (Miltenyi) (to 1:17.5 titer). GMP IL-2 (R&D Systems) (5001 U/mL from a 2100 IU/uL stock) Note: GMP IL-2 stored in the −80 C. Reconstitute and prepare according to manufacturer instructions.

Step (c). Media exchange to supplemented culture media (post-sorting) by centrifugation: Centrifugation in 4 C, at 300xG for 15 minutes, acceleration 7, deceleration 1.

Cells resuspension with supplemented culture medium to approx. 10×10⁶/ml. Manual cell count and viability of pulled sorted cells. Cell concentration adjustment to ~1.43×10⁶ viable cells/mL using supplemented culture medium. Cells transfer to a culture bag and incubation in a CO₂ incubator, 37 C (36 h-48 h). Sampling: Pre-Culture Fresh Treg for QC and research panel.

Step 1. Day 4. (step d) Treg Cryopreservation. Manual cell count and viability of cultured cells. Cells cryopreservation. Sampling: Pre-Cryo Treg for QC and research panel.

Step 2. Post—thawing. (step e) Day 1. Treg Thaw and Cell Culture: Preparation of supplemented culture medium: GMP TexMACS medium (Miltenyi). GMP OTC Human AB Serum HI GI (Access Biologics) (10% of total volume). GMP IL-2 (R&D Systems) (5001 U/mL from a 2100 IU/uL stock) Thawing and dilution of cryopreserved Treg cells. Manual count and viability.

Media exchange to supplemented culture media by centrifugation: Centrifugation in 4 C, at 300xG for 15 minutes, acceleration 7, deceleration 1. Cells resuspension with supplemented culture medium to approx. 10×10⁶/mL. Manual cell count and viability. Cell concentration adjustment to 1.43×10⁶ viable cells/mL. Cells transfer to an appropriate culture bag and overnight incubation in a CO2 incubator, 37 C. Sampling: Pre-Culture Thawed Treg for QC and research panel.

Step 2. Day 2. (step f) TransAct Addition. GMP TransAct (Miltenyi) addition (to 1:17.5 titer). Overnight incubation in a CO₂ incubator, 37 C.

Step 2. Day 3. Treg product ship out. Media exchange to Normosol-R+2% HSA by centrifugation: Centrifugation at 300xG for 15 minutes, acceleration 7, deceleration 1, in room temperature (RT).

Pre-Ship out Treg Drug Product testing. Ship out cells (4 C)/Immediate injections.

Sampling: Pre-Shipout Treg Drug Product for QC and research panel. Sample for Suppression assay (1-2.5*10\hat{ }6 cells). Store in 40 C fridge overnight.

Step 2. Day 4. (step g) Final Treg Product infusion and testing. Suppression assay set up. Post-Ship out Treg Drug Product research panel.

### Results.

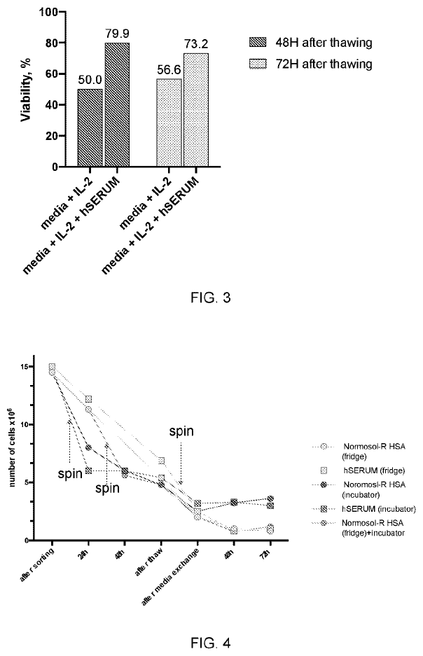

Small-scale process development runs with previously frozen products available at CTF. After thawing, different Treg products had a range of viability from 43% to 88% (FIG. 2) with overall cell loss from thawing to the end of the culturing (72 hours) more than 90%. Cells were CD3⁺, CD4⁺, FoxP3⁺, CD127^{low}, CD25^{low}, CD62^{low}. Using available products, a number of post-thaw experiments were set up to compare commercially available GMP media for T cell culturing and expansion suitable for protocol purposes and to determine optimal media supplementation. Based on those experiments TexMACS media (Miltneyi) was selected as a preferable media, if supplemented with IL-2 and human serum (10%). Addition of human serum dramatically changed viability of Tregs even if added to “no serum” culture for 24 hours (shipment step) (FIG. 3).

Engineering run to test pre-cryopreservation activation of Treg. Cell apheresis (step a), Treg selection (step b) and culturing/activation (step c) were performed to determine the effect of Treg activation before cryopreservation. This run showed significant improvement in cell viability, counts and normal Treg phenotype post-thaw and post-culture (CD3⁺, CD4⁺, FoxP3⁺, CD127^{low}, CD25⁺, CD62L⁺). Cell losses were seen over the media exchange steps (FIG. 4.). A modified approach by centrifugation without breaks was successfully tested in another engineering run.

Process development run (Full—N1). A full engineering run was performed using manufacturing protocol developed based on previously described process development and engineering runs. Final Treg product contained 63.1×10⁶ viable Treg cells (51.7% from post-sorting cell number) with viability 94.1% and normal Treg phenotype. Treg were shown to suppress responder cells proliferation.

Step 1. Leukapheresis product was successfully received (25 Jan. 2021). 80×10⁶ responder cells (in human serum+10% DMSO) and plasma were cryopreserved. CD25 selection went with no issues, enriched fraction diluted 1:1 and stored overnight at 40 C in bag. Staining and sorting went with no issues, purity >90%, Treg frequency of total events: 55% Cell count post media exchange showed no cell loss (see Table 1) Treg were pooled and cultured in TexMACS+10% GMP human serum, 5001 U/mL IL-2 and 1:17.5 TransAct. After 41 hours, cells underwent media exchange to 50% GMP human serum+50% cryo-mix and cryopreserved.

TABLE 1
Viability and count over manufacturing steps.

| | TOTAL | | VIABLE | COUNT |
|---|---|---|---|---|
| PRODUCT | COUNT | VIABILITY | COUNT | METHOD |
| Collection | 1.31E+10 | N/A | N/A | *Sysmex |
| CD25 Labeled Washed | 1.15E+10 | N/A | N/A | *Sysmex |
| CD25 Reduced | 9.90E+09 | N/A | N/A | *Sysmex |
| CD25 Enriched | 3.19E+08 | N/A | N/A | *Sysmex |
| Pre-Sort | 2.20E+08 | 94.20% | 2.07E+08 | Manual |
| Cell Sorter 1 | 5.85E+07 | N/A | N/A | *Influx |
| Cell Sorter 2 | 6.52E+07 | N/A | N/A | *Influx |
| Pooled Sorted Treg | 1.24E+08 | N/A | N/A | *Influx |
| Pre-Culture | 1.33E+08 | 91.90% | 1.22E+08 | Manual |
| Post-Culture | 1.11E+08 | 78.30% | 9.01E+07 | Manual |
| Post-Thaw | 7.14E+07 | 85.10% | 6.19E+07 | Manual |
| Pre-Culture | 6.08E+07 | 90.30% | 5.46E+07 | Manual |
| Post-Culture | 6.79E+07 | 94.10% | 6.31E+07 | Manual |
| Pre-Shipout | N/A | 90.70% | N/A | Manual |
| Post-Shipout | N/A | 89.90% | N/A | Manual |

Step 2. Treg thawed on 2 Feb. 2021, with ~35% observed cell loss due to cryopreservation. Culture media was prepared, cells were transferred to conical tube and bag was rinsed. Media exchange by centrifugation showed 14.85% cell loss. Cells were cultured in TexMACS+10% GMP human serum, 5001 U/mL IL-2. After −22 hours, 1:17.5 TransAct was added. After 44 hours of culture, cells underwent media exchange to Normosol-R+2% HAS. Final post-culture Treg viability was 94.10%, count was 6.31×10⁷ total viable cells which is about 52% of pre-culture cell number after sorting on Step 1.

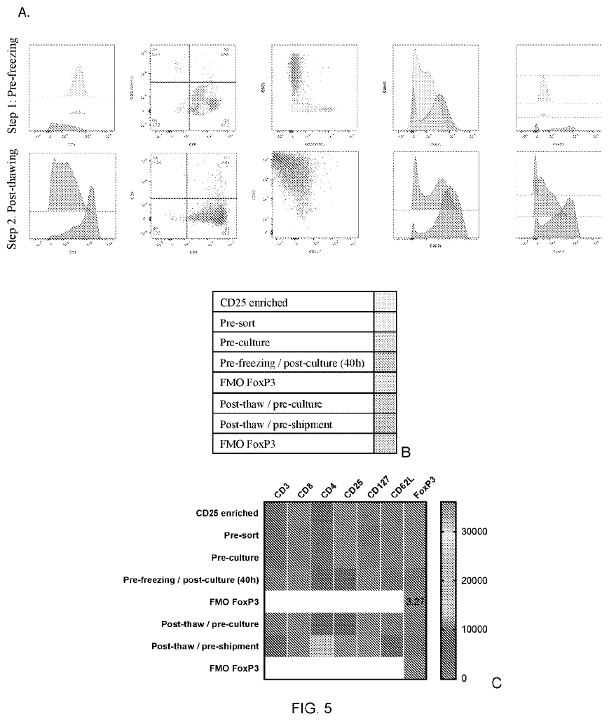

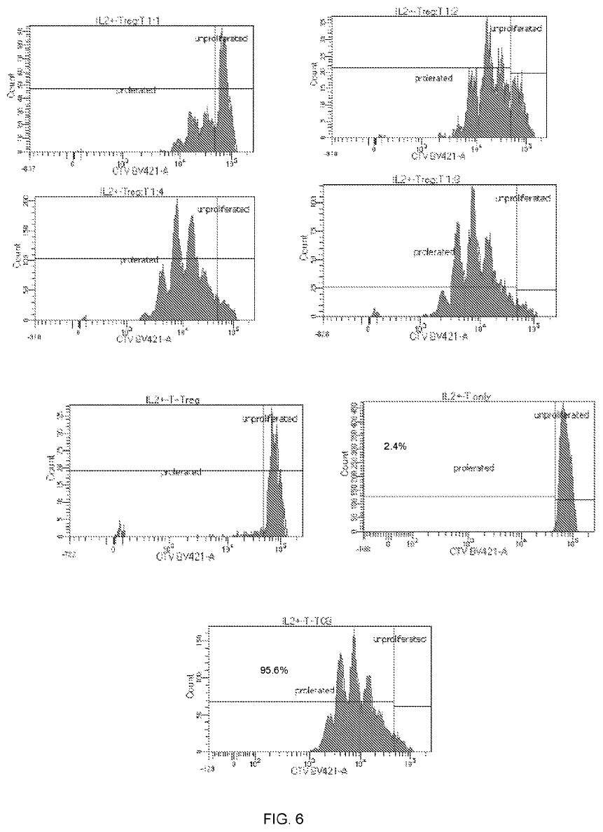

Research Flow Cytometry Panel. Research flow cytometry panel showed normal Treg phenotype (CD4⁺CD25⁺CD127^{low}FoxP3⁺) for Step 2 post-culture cells (see FIG. 5. Panel A. Cells profile on different manufacturing steps (dark blue—final product) Panel B. samples color-code and Panel C. geometric MFI heat map.) Negative control—responder cells without stimulation and responder cells without stimulation and with Tregs. Positive control—responder cells activated and without Tregs. Ratios of Treg to responders (1:1, 1:2, 1:4, 1:8) conditions had same number of responders (Tcon) and decreasing number of Tregs.

TABLE 2
Results from cell manufacturing qualification runs at Stanford CTF

| | | | FOXP3 | | |
|---|---|---|---|---|---|
| | | | expression in T | | |
| | | Viability of cells | regulatory cells in | | |
| | | in product for | product for | | Cell count |
| | | release (after | release (after | | adjusted for |
| | Cells count | cryopreservation, | cryopreservation, | | large volume |
| | after | thaw, | thaw, | | apheresis |
| | purification | resuscitation and | resuscitation and | | collection |
| | (12 L | preparation for | preparation for | Final Cell count | (30 L) for 70 |
| | apheresis) | infusion) | infusion) | (12 L apheresis) | kg recipient |
| Validation | 8.16 × 10⁷ | 92% | 80% | 4.0 × 10⁷ | 1.43 × 10⁶/kg |
| run 001 | | | | | |
| Validation | 4.62 × 10⁷ | 86% | 80% | 2.28 × 10⁷ | 0.81 × 10⁶/kg |
| run 002 | | | | | |
| Average* | 8.69 ± | 90 ± 3% | 80% | 4.36 ± | 1.56 ± |
| | 4.36 × 10⁷ | | | 2.28 × 10⁷** | 0.82 × 10⁶/kg |

*average and SD calculation is based on results of one process development run and two cell manufacturing validation runs (001 and 002).
**engineering run final products count based on post-stimulation sample

### Example 2

### Collection, Processing, and Shipping of Recipient Tregs Cellular Products

Patients enrolled in this study receive autologous Tregs in addition to donor cells as post-transplant therapy for kidney tolerance induction. All cell collections and processing to obtain purified Tregs take place at Stanford, as described below.

Patients on this arm of the study undergo non-mobilized peripheral blood apheresis well in advance of their kidney transplants. Patients undergo a single apheresis collection with a target total blood volumes of 20-30 L and the collected cells are used as the source of purified Tregs to be infused post-transplant. In the event a patient is unable to meet the collection target, a second collection may be performed. Each product collected is transferred to the Stanford BMT-CTF following fresh product transfer procedures. Products are maintained in secure storage at 2° C.-8° C. until processing begins. Samples are removed to assess cell count, viability, and Tregs content prior to purification.

The recipient's Tregs are purified using immunomagnetic selection and flowcytometric cell sorting. The cells undergo two rounds of activation by stimulating the CD3/CD28 expressing cells.

The first round is performed prior to cryopreservation of the Tregs and the second after thawing but prior to distribution of the cells for infusion.

The infusion of purified Tregs for recipients occurs within 24 hours of the donor HPC and Tcon infusion. Therefore, the recipient's cryopreserved Tregs are removed from Liquid N₂ storage and thawed by the CTF no more than 72 hours prior to the scheduled infusion in order to provide sufficient time for the second round of stimulation and product transport to the MCCT for patients treated at Northwestern.

Purification of autologous Tregs is by tandem selection as previously described, in which the cells are first enriched by CD25 expression by CliniMACS immunomagnetic selection followed by flow cytometric sorting for CD4⁺, CD127^{low/neg} expression. The resulting product is enriched for Tregs defined phenotypically as CD4⁺, CD25⁺, CD127^{low/neg} and expressing the transcription activator FoxP3. The latter is only detectable intracellularly, requiring fixation and permeabilization of the cells prior to staining and analysis by flow cytometry. Therefore, FoxP3 detection is used only to assess the percentage of Tregs in samples drawn from the final cellular product for infusion.

Cells recovered from the Tregs purification procedures are transferred to TexMACS medium (Miltenyi Biotec, Bergisch-Gladbach, Germany) supplemented with serum (Human GMP OTC AB Serum, Access Biologics, Vista, CA) and recombinant human IL-2 (R&D Systems, Minneapolis, MN). T Cell TransAct™ reagent (Miltenyi Biotec) is added to the Tregs suspension as the stimulatory agent and the product placed at 37° C., 5% CO₂ for 24-48 hours.

Materials for Stimulation of Autologous Tregs: TexMACS GMP Medium (Miltenyi Biotec, Bergisch-Gladbach, Germany); T Cell TransAct™; human MACS GMP Cell Differentiation Bags; Human GMP OTC AB Serum (Access Biologics, Vista, CA); Normosol-R, pH 7.4; Recombinant human IL-2 (R&D Systems, Minneapolis, MN). The cells will be separated from the stimulation medium by centrifugation and resuspended in serum (Human GMP OTC AB Serum, Access Biologics, Vista, CA). After freezing, the cells are placed in LN₂ vapor phase storage until thawed in advance of release for infusion.

Materials for Cryopreservation of Autologous Tregs: Normosol-R, pH 7.4; Hydroxyethyl Starch (6% Hetastarch); Human Serum Albumin (HSA); Dimethyl Sulfoxide (DMSO); CryoMACS Freezing Bag.

Cryopreserved Treg products are thawed and a second round of stimulation performed prior to release of the Tregs for infusion. Thawing occurs less than 72 hours prior to infusion and the cells are placed in TexMACS medium supplemented with human serum (human, GMP OTC AB Serum, Access Biologics, Vista, CA), and recombinant human IL-2 R&D Systems, Minneapolis, MN), as in Step 1 of the stimulation process. The cells are placed at 37° C., 5% CO₂ to stabilize the cells prior to stimulation. After 12-24 hours, TransAct™ is added to the Treg suspension to initiate stimulation and the cells returned to 37° C., 5% CO₂ for up to 24 hours, after which the cells are separated from the stimulation medium by centrifugation and resuspended in Normosol-R, pH 7.4 (Hospira, Lake Forest, IL) supplemented with 5% HSA (Baxalta, Los Angeles, CA) as final preparation for infusion. After samples are drawn for release testing, the final Tregs product are maintained in secure, monitored conditions at 2°−8° C. until its final transfer for infusion. Transfer of the Tregs product to the infusion site is as described below.

Samples of the final Tregs product are aseptically drawn and submitted for release testing. The testing and criteria for release are listed below. Any reduction in the cell count for infusion to prevent exceeding the maximum dose of Tregs is based on the recipient's most recent weight and any excess cells will be removed from the product prior to release. For products to be shipped to the MCCT, excess cell removal shall be performed by the CTF as preparation for transport and will not be performed by the MCCT.

### Release Testing and Criteria for Autologous Tregs

| Assay | Method | Criterion |
|---|---|---|
| Tregs dose | Hemocytometer | ≤5 × 10⁶ CD45⁺, CD4⁺, FoxP3⁺ |
| | cell count | cells/kg (recip. body wt.) |
| Viability | 7-AAD or equivalent | ≥70% viable cells |
| | exclusion dye | |
| Phenotype | Flow cytometry | ≥80% CD45⁺, CD4⁺, FoxP3⁺ |
| Sterility: | Gram staining | Negative |
| Endotoxin | EndoSafe PTS | ≤5 EU/kg (recip. body wt.) |

Parallel product sterility testing of product samples drawn at the same time as those submitted for Gram staining is performed using standard 14-day cultures for detection of both aerobic and anaerobic microorganisms. However, as the culture testing will not be completed prior to distribution of the cells for infusion, they are supplementary to the criteria for product release. Upon detection of any microbial growth in the 14-day cultures, the testing laboratory shall immediately notify the CTF and the Plan of Action for the Infusion of Microbially Contaminated Products shall be initiated as follows. Upon notification of detectable growth in the product sterility culture, the CTF shall immediately contact the attending physician or designee. The report will be communicated to the study principal investigator (PI) and co-investigators, the cell processing laboratory directors, and the research coordinators. The identity and antibiotic susceptibility of the contaminant shall be determined and reported to the principals as indicated above. The product recipient will be monitored for any signs of infection and blood cultures initiated. Upon evidence of infection, either through clinical symptoms or blood culture testing, the recipient will be administered appropriate antibiotic therapy. The recipient will be continually monitored and the efficacy of the therapy assessed. Therapy will be modified as deemed necessary until the infection is cleared.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of cryopreserving regulatory T cells (Tregs) for enhanced viability, cell recover and function, the method comprising:
performing in a period of less than about 72 hours pre-freezing steps of:
(a) collecting cells from a human donor;
(b) selecting for Tregs;
(c) activating Tregs in vitro;
(d) cryopreserving Tregs; and
performing in a period of less than about 72 hours post-thaw steps of:
(e) thawing Tregs,
(f) activating Tregs in vitro.

2. The method of claim 1, wherein the Tregs are collected from the human donor by apheresis, and are selected for the phenotype of CD4⁺ CD25⁺ CD127^{lo}.

3. The method of claim 1, wherein the apheresis collection is enriched by selection for CD25⁺ cells.

4. The method of claim 1, wherein the Tregs are activated in step (c) for a period of from 24 to 48 hours.

5. The method of claim 1, wherein the Tregs are activated in step (c) in medium comprising human serum at a concentration of from about 5% to about 15%.

6. The method of claim 1, wherein the Tregs are activated in step (c) in medium comprising IL-2.

7. The method of claim 1, wherein the Tregs are activated in step (c) in medium comprising a T cell activating agent.

8. The method of claim 1, wherein the Tregs are activated in step (f) for a period of from about 12-24 to 48 hours in the absence of a T cell activating agent, and for a period of from about 12 to 24 hours in the presence of a T cell activating agent.

9. The method of claim 1, wherein the Tregs are activated in step (f) in medium comprising human serum at a concentration of from about 5% to about 15%.

10. The method of claim 1, wherein the Tregs are activated in step (f) in medium comprising IL-2.

11. The method of claim 1, further comprising the step of:
(g) administering the Tregs of step (f) to a recipient in need thereof.

12. The method of claim 11, wherein the composition comprising Tregs are administered at a dose of from 10⁴ cells/kg body weight to about 10⁹ cells/kg body weight.

13. The method of claim 11, wherein the composition comprising Tregs comprises at least about 50% CD4⁺ CD25⁺ CD127^{lo} cells.

14. The method of claim 1, wherein said administering comprises infusing into said human subject said composition comprising Tregs.

15. The method of claim 1, wherein said composition comprising Tregs is autologous relative to said human subject.

16. The method of claim 1, wherein said composition comprising Tregs is allogeneic relative to said human subject.

17. A composition comprising a population of Tregs obtained by the method according to claim 1.

18. A composition comprising a population of cryopreserved Tregs obtained by the method of steps (a) to (d) of claim 1.
